(19) United States
(12) Patent Application Publication
(10) Pub. No.: US 2024/0304340 A1
(43) Pub. Date: Sep. 12, 2024

(54) COMPUTERIZED METHODS AND APPARATUS FOR FACILITATING THE PRESCRIBING OF PHARMACEUTICALS

(71) Applicant: NEXTGEN MANAGEMENT LLC, Boca Raton, FL (US)

(72) Inventors: Ezra Hanz, Boca Raton, FL (US); Jeffrey Arthur Stahl, Boca Raton, FL (US); Lewis Arnold Stahl, Boca Raton, FL (US)

(21) Appl. No.: 18/407,124

(22) Filed: Jan. 8, 2024

Related U.S. Application Data
(60) Provisional application No. 63/437,534, filed on Jan. 6, 2023.

(51) Int. Cl. G16H 80/00; G16H 40/20

(52) U.S. Cl. CPC G16H 80/00; G16H 40/20

(57) ABSTRACT

This disclosure pertains to computer-assisted methods, apparatus, and systems for providing information about pharmaceuticals and other medications to healthcare providers to assist in the prescribing of such medications to patients.

### FIELD OF THE INVENTION

This disclosure pertains to computer-assisted methods, apparatus, and systems for providing information about pharmaceuticals and other medications to healthcare providers to assist in the prescribing of such medications to patients.

### BACKGROUND

The vast majority of diseases and medical conditions today are treated via the prescription of pharmaceuticals and/or other medications. In most countries, there are laws requiring that many of these medications cannot be obtained by an individual without a prescription for such from an appropriate healthcare professional. In the United States, for instance, many pharmaceuticals and other medications (hereinafter, collectively, medications or drugs) must be prescribed by a qualified medical practitioner, such as a medical doctor (M.D.), a nurse practitioner (NP), a Doctor of Osteopathic Medicine (D.O.), or a physician's assistant (P.A.) (hereinafter, health care providers or HCPs). Novel medications and novel applications for existing medications are being developed at a rapid pace, such that it may be difficult for HCPs to remain fully informed and up to date on all such medications. Accordingly, pharmaceutical companies and the like employ many persons, whose jobs it is to stay highly informed about certain medications and to talk to HCPs to help the HCPs stay informed about the latest medications and uses for medications. For instance, each pharmaceutical company typically employs (or otherwise contracts with) many pharmaceutical representatives (hereinafter, Rep or PRs) to visit HCPs on a regular basis to inform them about one or more medications offered by the company. Each Rep commonly is assigned a particular geographic area to cover and a particular set of medications on which the Rep is trained and knowledgeable.

Many countries have enacted laws, rules, and/or regulations that tightly control the types of information and/or ways a Rep can convey such information to an HCP. Violations of such laws by pharmaceutical companies and/or their Reps can carry heavy fines and other adverse effects, and should be avoided at all costs.

In addition, HCPs tend to be extremely busy and often do not have the time to meet in person with all the Reps for the various pharmaceutical companies. Yet further, the electronic age as well as a global pandemic have made in person meetings with pharma Reps even less appealing to many HCPs. Accordingly, it has become increasingly common for such information to be exchanged between Reps and HCPs via electronic means, such as telephone and email.

Since improper, incorrect, and misunderstood information about a medication can lead to an HCP issuing an improper or incorrect prescription for a patient, which, in turn, could result in potentially serious adverse consequences for patients, it is extremely important to assure that information about medications is correctly and properly conveyed from Reps to HCPs and that a detailed record of the communications between PRs and HCP is maintained.

### SUMMARY

In an embodiment, a computer-readable device for a pharmaceutical representative (PR) to interface with a health care provider (HCP) via electronic communications about a medication comprises non-transitory instructions, which, when executed by a processor, cause the processor to perform operations, the operations comprising; receiving an electronic communication identifying an HCP, a medication, and an electronic address for communicating with the HCP; responsive to receiving the electronic communication, generating a first graphical user interface (GUI) configured for scheduling an electronic meeting between the HCP and a PR, the first GUI comprising at least (1) a field for populating with an identity of the PR, (2) a field for populating with an identity of the HCP, (3) a field for populating with the medication, (4) a field for populating with a time for a meeting between the PR and the HCP; responsive to at least each of the four fields being populated, transmitting to the HCP an invitation to an electronic meeting between the HCP and the PR; creating an electronic meeting environment in which the PR and HCP may conduct the scheduled electronic meeting, the environment including at least capabilities for audio and video communication between the HCP and the PR via a communication network, capability for uploading files for the meeting, and file sharing capabilities; recording the meeting, including recording at least audio of the meeting, video of the meeting, files uploaded in the meeting, files downloaded by the HCP in the meeting, and an identity of each participant in the meeting; storing in a database at least the time of the meeting, a duration of the meeting, an identity of the medication, the participants in the meeting, the files uploaded in the meeting, and the files downloaded by the HCP in the meeting; and searching the database by any one or more of the fields.

In another embodiment, a computer-readable device for a pharmaceutical representative (PR) to interface with a health care provider (HCP) via electronic communications about a medication comprises non-transitory instructions, which, when executed by a processor, cause the processor to perform operations, the operations comprising: generating a graphical user interface in which a health care provider (HCP) may select a medication and request to be contacted by a pharmaceutical representative (PR) about the medication; responsive to the request, searching a first database of PRs and the medications the PRs are permitted to represent to identify a PR suitable to the request as a function of at least the selected medication; generate an electronic communication identifying the requesting HCP and the selected medication; and transmitting the electronic communication to the identified PR.

In yet another embodiment, a method for a pharmaceutical representative (PR) to interface with a health care provider (HCP) about a medication via electronic communications comprises: receiving an electronic communication identifying an HCP, a medication, and an electronic address for communicating with the HCP; responsive to receiving the electronic communication, generating a first graphical user interface (GUI) configured for scheduling an electronic meeting between the HCP and a PR, the first GUI comprising at least (1) a field for populating with an identity of the PR, (2) a field for populating with an identity of the HCP, (3) a field for populating with the medication, (4) a field for populating with a time for a meeting between the PR and the HCP; responsive to at least each of the four fields being populated, transmitting to the HCP an invitation to an electronic meeting between the HCP and the PR; creating an electronic meeting environment in which the PR and HCP may conduct the scheduled electronic meeting, the environment including at least audio and video communication between the HCP and the PR via a communication network, capability for uploading files for the meeting, and file sharing capabilities; recording the meeting, including recording at least audio of the meeting, video of the meeting, files uploaded in the meeting, files downloaded by the HCP in the meeting, and an identity of each participant in the meeting; storing in a database at least the time of the meeting, a duration of the meeting, an identity of the medication, the participants in the meeting, the files uploaded in the meeting, and the files downloaded by the HCP in the meeting; and searching the database by any one or more of the fields.

### BRIEF DESCRIPTION OF THE DRAWINGS

A more detailed understanding may be had from the detailed description below, given by way of example in conjunction with the drawings appended hereto. Figures in such drawings, like the detailed description, are exemplary. As such, the Figures and the detailed description are not to be considered limiting, and other equally effective examples are possible and likely. Furthermore, like reference numerals (“ref.”) in the Figures (“FIGs.”) indicate like elements, and wherein:

FIG. 1 is a block diagram providing a high-level illustration of the main components of a system for allowing physicians and other HCPs to contact and interact with pharmaceutical representatives in accordance with embodiments;

FIG. 2 is a graphical user interface (GUI) page of an electronic health records software suite into which embodiments may be integrated;

FIG. 3A is an exemplary graphical user interface for presenting a list of medications to an HCP and for the HCP to request samples thereof in accordance with embodiments;

FIG. 3B is another exemplary graphical user interface for allowing a user to enter the name of a medication in accordance with embodiments;

FIG. 4 is an exemplary graphical user interface that may be presented to HCP after a drug is selected providing basic information about the selected drug in accordance with embodiments;

FIG. 5 shows an exemplary text message that may be generated by the HPIS for an HCP to request to be contacted by a Rep in accordance with embodiments;

FIG. 6 is an exemplary graphical user interface that may be presented to a pharmaceutical representative for reviewing previously scheduled meetings and for adding new meetings in accordance with embodiments;

FIG. 7 is a graphical user interface for scheduling a video conference between an HCP and a Rep in accordance with some embodiments;

FIG. 8 is an example of an email that may be sent to each of the invitees of a meeting created and scheduled in accordance with embodiments;

FIGS. 9A and 9B shows an example of a detailed report of a video conference between an HCP and a Rep in accordance with some embodiments; and

FIG. 10 is a flowchart showing a process in accordance with some embodiments.

### DETAILED DESCRIPTION

In the following detailed description, numerous specific details are set forth to provide a thorough understanding of embodiments and/or examples disclosed herein. However, it will be understood that such embodiments and examples may be practiced without some or all of the specific details set forth herein. In other instances, well-known methods, procedures, components, and circuits have not been described in detail, so as not to obscure the following description. Further, embodiments and examples not specifically described herein may be practiced in lieu of, or in combination with, the embodiments and other examples described, disclosed, or otherwise provided explicitly, implicitly, and/or inherently (collectively “provided”) herein.

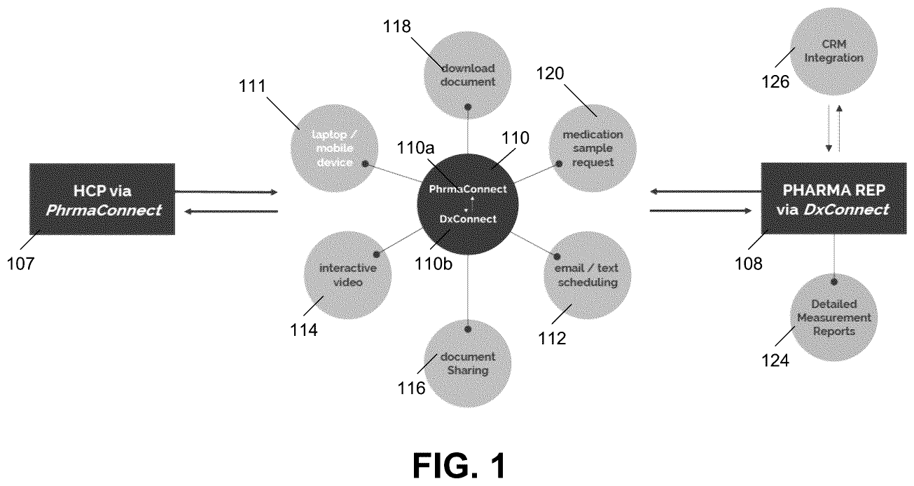

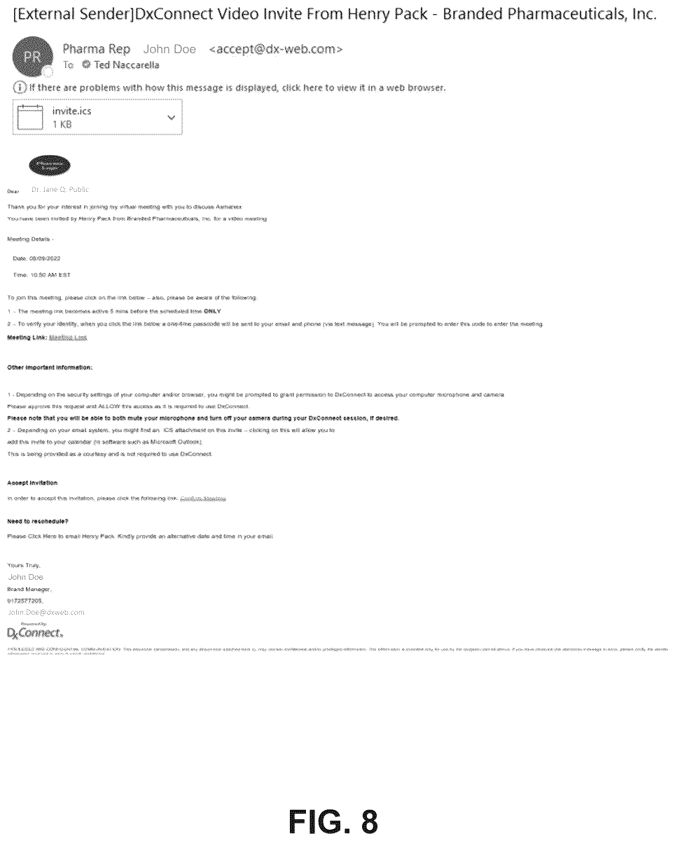

FIG. 1 is a block diagram providing a high-level illustration of the main components of a system for allowing physicians and other HCPs to contact and interact with pharmaceutical representatives in a web-based environment that maintains records of the interactions, allows for video conferencing, and allows for exchange of documents, videos, photographs, and other written information (such as drug information sheets, marketing materials, etc.). In some embodiments, the records of the interactions, discussed in more detail below in connection with FIG. 8, are made available to the Reps. Such records may be used at a future time to establish compliance (or non-compliance, as the case may be) with any laws, regulations, or rules applicable to Rep communications with HCPs.

Physicians and other HCPs write billions of prescriptions each year. Adverse drug events are known to decline when physicians electronically prescribe medication because physician handwriting is sometimes illegible to pharmacists. Moreover, the writing of prescriptions by unauthorized persons continues to be a problem because the current systems are easily duplicated. These shortcomings have contributed to rising health care and drug prescription costs. These challenges have led to an increased use of electronic prescription systems and, more generally, electronic medical records (EMR) for the management of patient files.

Electronic prescription dispensing systems (EPDSs), such as the DxScript electronic medical prescription system available from NextGen Management LLC, typically include the steps of entering patient information electronically into an electronic health record (EHR) system. The prescriber enters the patient information and necessary prescription information, such as drug and dosage, into the EMR system via a suitable graphical user interface (GUI) as known in the art. U.S. Pat. No. 11,049,597, herein incorporated by reference in its entirety, describes one such medical prescription system.

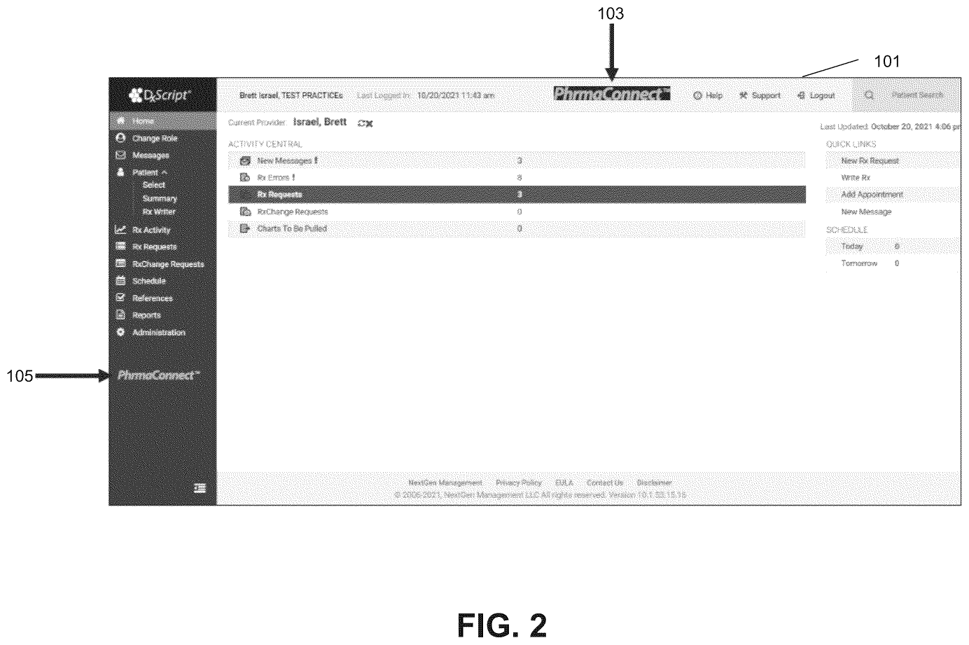

A system such as shown in FIG. 1 (hereinafter termed an HCP/PR interface system or HPIS) 110 may be integrated into or otherwise interfaced with (e.g., through an application programming interface (API)), an EPDS or other EHR so that an HCP can directly access the features thereof while preparing a prescription for a patient in the EPDS or other EHR system. In some embodiments, access to the HCP/PR interface system may be integrated into or made accessible from an EHR by providing a clickable icon on one or more user interfaces (e.g., web pages) of the EHR that links to the HPIS, such as illustrated by FIG. 2.

Particularly, FIG. 2 shows a graphical user interface (GUI) page 101 within the aforementioned DxScript EPDS. The illustrated page essentially is a start (or Home) page in the DxScript EPDS. An HCP may click on either of the PhrmaConnect buttons 103 or 105, which buttons may be hyperlinks to a home page of the HPIS system 110 of FIG. 1. In an embodiment, the HPIS may be comprised of two distinct software programs (suites), namely, (1) a first program (herein termed PhrmaConnect) that is responsible for interfacing with the HCP (either directly or through the HCPs EHR or EPDS software, such as described herein) to generate an initial contact between the HCP and an appropriate Rep, and (2) a second program (herein termed DxConnect) to manage and record all remaining electronic interactions between the HCP and the PR. Thus, the system is flexible in that, if the PR is contacted by an HCP via PhrmaConnect, but wishes or needs to use a different video conferencing system than DxConnect, that is possible (although not recommended for, at least, security and confidentiality reasons, as will be discussed further below).

When entering the HPIS from this point, the HCP has not yet selected a medication of interest. Therefore, in an embodiment, the hyperlinks 103, 105 on this page of the EPDS may be programmed to lead to a list of one or more “featured” medications that the HCP may select from. The featured medications list may, for example, comprise any one or more of (1) medications that are the most frequently prescribed medications, (2) medications that are the most frequently asked about (either in general or based on historical data from the HPIS itself (possibly using any of the geographical factors mentioned further below in this paragraph), (3) newest or most recently approved medications (or uses of medications), (4) sponsored medications (e.g., medications that pharmaceutical companies have provided consideration to have listed), and (5) medications dictated by current events (e.g., medications relevant to treatment of a local outbreak of a particular disease). Any of the above criteria may be made further dependent on the geographic location of the requesting HCP, wherein such geographic dependency may be based on country, state, city, zip code, or any other sized geographic area, e.g., the most prescribed medications in the zip code in which the HCP is located.

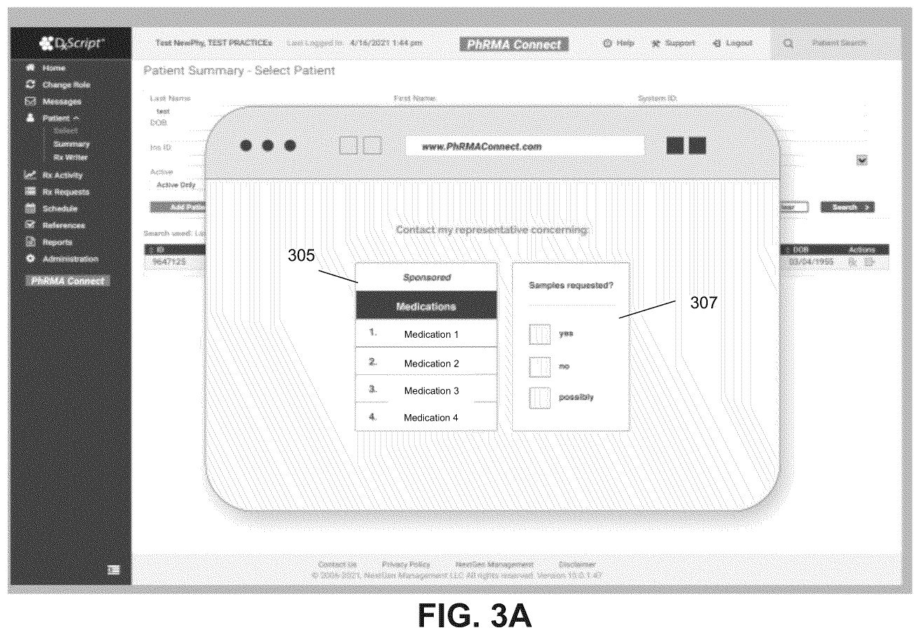

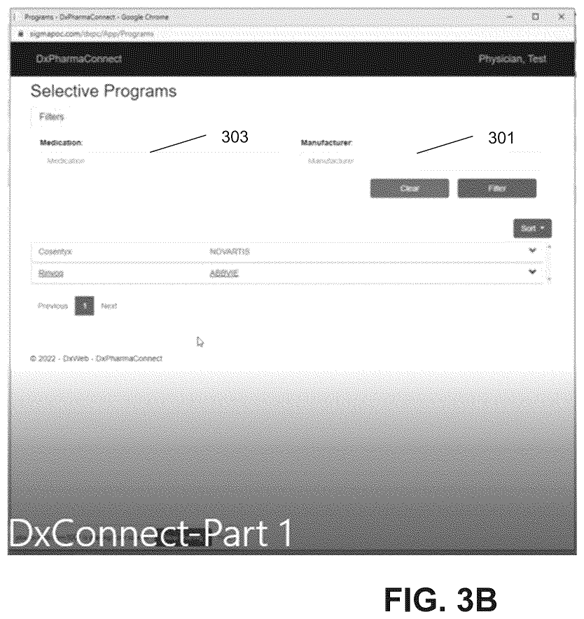

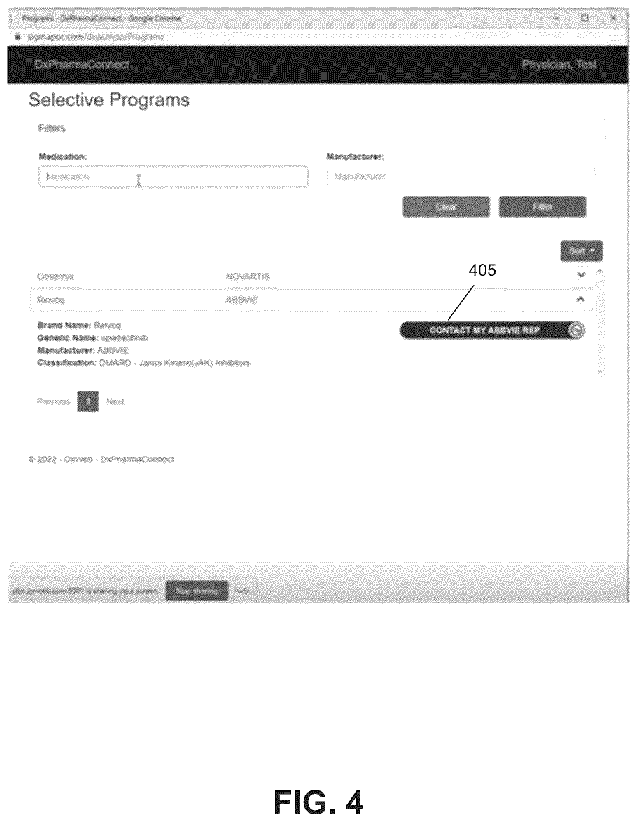

As noted above, one of the primary functions of the HPIS is to initiate contact between the HCP and an appropriate PR when the HCP desires information about a particular medication. For instance, in some embodiments, the clickable icon may appear only on pages of the EPDS system in which the HCP has already made a choice of a particular medicine that is of interest to the HCP (e.g., to prescribe to a patient). In such embodiments, the hyperlink may lead to a different page of the HPIS (as opposed to the aforementioned featured medication page, which would likely include a list of medications that are irrelevant to the HCP's immediate concerns). In such embodiments, for instance, the hyperlink may be programmed to lead to a page corresponding to the already selected medication (see, e.g., FIG. 4, described in more detail below) or to a page in which the HCP may type the name of the medication of interest into a text entry box (see FIG. 3B, described below in further detail).

In other embodiments, clicking on the icon 103 or 105 might first cause a drop-down menu or pop-up menu to be displayed. That menu may include the aforementioned list of featured medications. FIG. 3A shows an example of such a list of featured medications 305, along with a box 307 in which the user may select from three options about whether the user want samples of the selected medication(s), namely, Yes, No, and Possibly. The HCP may select one (or more) of the featured medications and click the desired option in box 307.

FIG. 3B shows an alternative embodiment of a pop-up screen that may be presented when an HCP clicks on icon 103 or 105 in FIG. 2. In this embodiment, the pop-up box includes a text entry box 303 into which the HCP may type the name of the drug of interest. In a preferred embodiment, as letters are entered a list of drugs starting with those letters is presented in a drop-down menu from the text entry box so that the HCP can stop typing when he sees the name of the drug of interest in the drop-down menu and then click on that name to complete the text entry. The pop-up GUI alternately or additionally may include a list of medications (e.g., in alphabetical order) that can be scrolled through to select the drug of interest.

Furthermore, there may be a text entry box for entering the name of a manufacturer of medications. In a preferred embodiment, as letters are entered, a list of manufacturers starting with those letters is presented in a drop-down menu from the text entry box so that the HCP can stop typing when he sees the name of the manufacturer of interest in the drop-down menu and then click on that name to complete the text entry. When a drug manufacturer is selected, the list of drugs 305 is modified to show only the drugs offered by that manufacturer.

After a medication is selected (e.g., via the user interface of FIG. 3A or FIG. 3B), the HCP may be presented with a screen such as shown in FIG. 4 providing basic information about the drug, such as the name of the drug, the generic name of the drug, the manufacturer of the drug, and the classification of the drug. In addition, a radio button 405 may be provided that can be clicked to indicate that the HCP wishes to be contacted by a Rep about the selected drug.

The HPIS maintains a database of the names, locations, NPIs (National Provider Identification numbers) and other relevant information about the HCPs using its system. This information may be available from the EPDS(s) to which it interfaces or may be maintained separately.

Once the HCP has selected a particular medication and/or clicked on the radio button 405, the HPIS searches a database to identify an appropriate PR for that medication. The database is maintained so as to have an up-to-date list of PRs categorized by any one or more of name, contact information, pharmaceutical company(ies) that they represent, medications that they represent, and geographical territory(ies) for which they are responsible (e.g., by zip codes) The HPIS searches the database to identify the appropriate PR based on the selected drug and the known location (e.g., zip code) of the requesting HCP.

Once the HPIS has determined the appropriate PR, it may present the HCP user with information about that selected PR. In other embodiments, it may not. In some embodiments, it will take the user to a new GUI in the HPIS system. That page may take many forms. For instance, in one embodiment, it may be as simple as a page informing the HCP that a suitable PR has been informed of the HCP's inquiry and that the PR will be contacting the HCP soon. In other embodiments, it may include information about the particular PR, such as any one or more of name, company represented, address, email, telephone number, etc.

In other embodiments, the system may allow the HCP to contact the PR directly. For instance, it may generate and display to the HCP a draft email or text message or other electronic communication that the HCP can send to the PR. In one such embodiment, the HPIS may interface with the email software and/or text messaging software of the user's computer or mobile device to generate and present the user with a draft email and/or text message to the selected PR. In other embodiments, the email or text functionality may be included in the HPIS.

In some embodiments, the user may be permitted to customize the email or text message. In other embodiments, that may not be permitted. In yet other embodiments, the HCP may be presented with a set of preconfigured options of the message to select from that may be sent to the PR. For instance, the HCP may be given the option (e.g., via a radio button) to request samples of the medication directly within the message that is sent to the PR.

In other embodiments, the HCP is not presented with a draft email or text message or even informed of the identity of the selected PR, but, instead, is merely informed that a PR has been (or will be) contacted, and, e.g., that the PR should be contacting the HCP soon. This type of embodiment may be preferable because it is the simplest for the HCP, allows greater uniformity of initial contact, and allows all actual substantive communications between the HCP and the Rep to be effected by and managed within a single software program (i.e., the DxConnect portion of the suite mentioned above and described in detail below), rather than be split between the two portions of the HPIS system (PhrmaConnect and DxConnect).

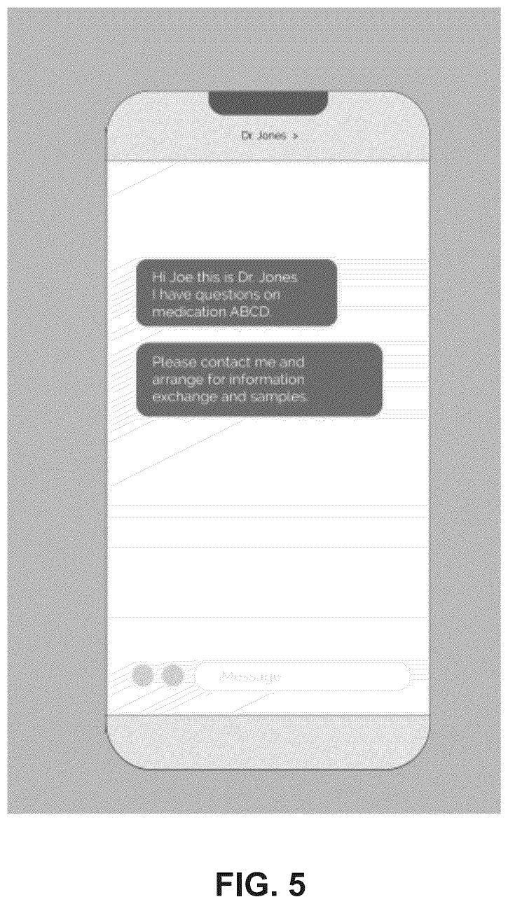

FIG. 5 shows an exemplary text message generated by the HPIS that may be sent to the selected appropriate Rep responsive to a request for information about a medication made via the PhrmaConnect software.

As noted above, in at least one preferred embodiment, the HCP does not see this message (as the HPIS may generate and send the message directly to the PR without HCP involvement).

In other embodiments mentioned above, this message may be presented to the HCP for sending by the HCP himself or herself and may even allow the HCP to customize the message.

In either case, the PhrmaConnect software sends the text message (and/or email) to the P. In an embodiment, the PhrmaConnect software may interface with a preexisting email program, such as Outlook, to send the email to the HCP. However, in a preferred embodiment, the PhrmaConnect software includes the text message and/or email functionality itself, rather than interfaces with another email or text messaging system. In yet other embodiments, the PhrmaConnect software suite may interface with the DxConnect software suite (discussed in more detail below) and allow the DxConnect software suite to generate and/or send the message(s). Keeping the functionality within the HPIS (whether the PhrmaConnect portion or the DxConnect portion) allows greater control over all aspects of the communications between the HCP and Rep.

The email or text may be received (by the PR) on any email or text platform. However, in one preferred embodiment, the email address (or text address) for the Rep is a unique email address assigned within the DxConnect portion of the HPIS and that is received within the DxConnect software suite such that all electronic communications between the HCP and the Rep can be recorded directly within the HPIS, including the initial contact communication.

In an embodiment, as previously noted, the second portion of the HPIS, the DxConnect portion manages all further interactions between the HCP and Rep.

When the PR receives the email or text message (hereinafter, collectively, “notice”), the PR may text or email the requesting HCP to schedule a meeting between the PR and the HCP. Preferably, these exchanges are all made using email and/or texting functionality built directly into the DxConnect software (as described more fully hereinbelow), and a record of them is kept in the HPIS system.

In embodiments, the functionalities of the PhrmaConnect and Dx Connect software suites could be contained within a single software package. However, one advantage of having them as separate independent modules is so that the HCP may request to be contacted by a PR extremely easily via a software interface that is incorporated directly into the HCP's EPDS (the PhrmaConnect portion). However, every other communication between the HCP and the PR is conducted within the second software suite (DxConnect), which is solely under the control of the PR (i.e., no portion of this software is in the possession of the HCP). In other words, the PhrmaConnect portion of the HPIS is separate from the DxConnect portion primarily so that the HCP has ready access to software that makes it easy and convenient to initiate the requests to the Rep via the PhrmaConnect portion of the system that is directly incorporated within the HCP's EPDS or EHR software suite. However, all other functionality of the HPIS is contained in the DxConnect portion of the software. Thus, once the request is generated, the DxConnect software can take over.

In any event, when the PR receives the email or text, the PR may open a file in DxConnect corresponding to this request for information (into which all interactions between the HCP and PR will be recorded as discussed in more detail below). In some embodiments, the file opening may be caused to occur automatically upon the PR receiving the message in DxConnect. In other embodiments, the file opening may be caused to occur automatically only upon the PR creating a meeting invitation corresponding to the request in the DxConnect software (as will be described in more detail below). A copy of (or link to) the email and/or text message may be kept in the file.

The DxConnect software also comprises full video conferencing software functionality, including scheduling of the video conference (including interfacing with external calendaring software suites, such as Outlook, to add the video conference to the users' calendars, email and/or texting functionality to send an electronic invitation to the requesting HCP, and video conferencing functionality for conducting the meeting (including full audio and video recording capabilities, including, for instance, screen sharing, document sharing, videos, pictures, etc.).

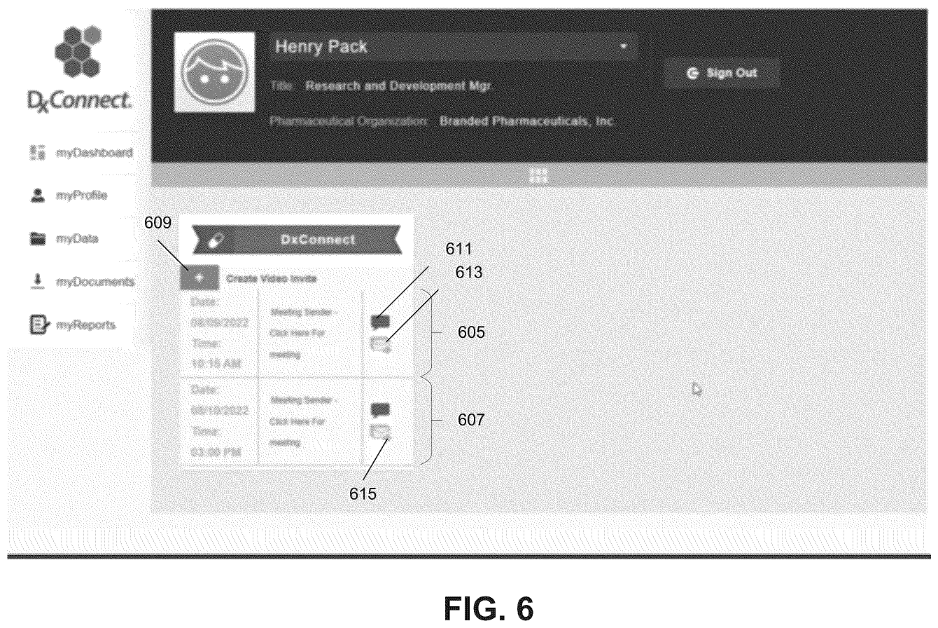

FIG. 6 shows an exemplary GUI that the DxConnect suite may present to the PR for scheduling a video conference with the HCP. As seen in this example instance of the GUI, it shows information about previously scheduled meetings between the particular PR and the particular HCP, 605, 607, and also includes a radio button 609 for scheduling a new meeting. As can be seen, each item 605 or 607 pertaining to a previously scheduled meeting shows the date and time of the meeting, the name of the PR (generically shown as “Meeting Sender” in FIG. 6., but which would normally comprise an actual name or ID number of the PR), and includes two radio buttons, 611, 613 for causing a text (611) or an email (613) invitation to the meeting to be sent to the prospective participants for that meeting. In embodiments, the radio buttons may be caused to change appearance after the invitation is sent to differentiate between meetings for which the invitations have been sent and meeting for which the invitations have not yet been sent. For example, an arrow 615 may be added to the relevant icon after the meeting invitation has been sent via the corresponding medium (e.g., email or text).

As will be discussed in more detail below for each of the meetings that has occurred in the past (e.g., meetings 605 and 607), the Dx Connect software maintains a record of the scheduled meeting (e.g., an audiovisual recording of the meeting) and all emails and texts between the PR and the HCP relevant to that meeting in the aforementioned file corresponding to that meeting in the DxConnect Software, which record may be accessed by mouse clicking in an appropriate area of the display corresponding to that meeting (e.g., the text “Click here for meeting”).

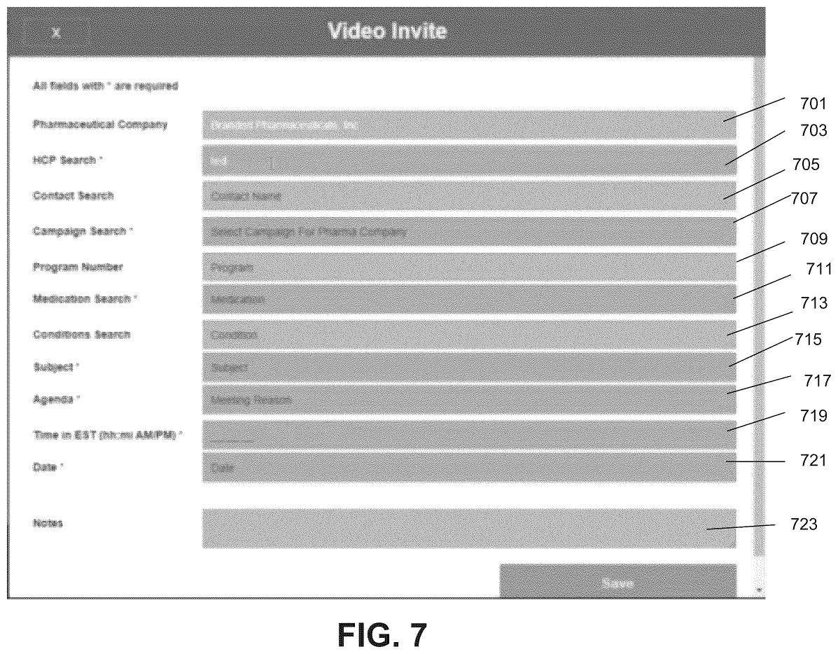

Turning back to the topic of scheduling a new meeting, FIG. 7 is an exemplary GUI that may be presented to the PR upon clicking the button 609 for creating a new invitation. As seen in FIG. 7, the GUI may include:
a field 701 for filling in the name of the pharmaceutical company that the PR represents with respect to the medication of interest;
a field 703 for filling in the name of one or more of the requesting HCPs. Note that there may be more than one individual at a given practice that wishes to be on the video call);
a field 705 for filling in the name of any other PR that it may be desirable to have on the video call;
a field 707 for filling in a particular marketing campaign of the relevant pharmaceutical company relating to the selected medication. This field might be the name of the particular medication of interest or a particular application of the medication or a particular dosage of the medication, etc.;
a field 709 for filling in an alphanumeric identification of the relevant campaign, if applicable. Particularly, pharmaceutical companies often have a specific alphanumeric ID for each of its advertising/sales campaigns;
a field 711 for filling in the name of the medication. The data this field may be populated with the same name as in field 707. Also, this field may have the option of different dosages of the relevant medication;
a field 713 for filling in any medical condition that may be relevant to the discussion (e.g., asthma),
a field 715 for filling in the subject of the meeting. This may be a freeform field that can be filled in with anything (and, often, may just be a repeat of another field, such as the name of the medication to be discussed (i.e., field 711), the name of the condition to be discussed (i.e., field 713), etc.);
a field 717 for filling in the agenda of the meeting. This field may be limited to selections from a dropdown menu insofar as one may wish to search for record files pertaining to specific agenda types. In one example, the choices for this field may be limited to options such as Initial meeting, Follow up meeting, Demo, Medication Update, New Medication Indication, Medication Dosage Update, New Medication Approval, and Important Medication Research Findings;
a field 719 for filling in the time of the meeting;
a field 721 for filling in the date of the meeting; and
a field 723 for filling in any additional notes that the Rep may deem relevant to the meeting.

Any one or more of these fields may be made searchable, e.g., meaning that one can start typing in that field and a list of the possible options meeting the typed characters will be presented (and narrowed down as the user continues to type in additional characters).

In some embodiments, in one or more of the data entry fields (e.g., medication, campaign, pharmaceutical company, HCP), the user may be limited to selecting only from a preconfigured list of options.

In some embodiments, the fields that are required are initially shown in a different manner that the optional fields, e.g., in red, whereas optional fields are shown in grey. In some embodiments, the red fields may turn a different color once they are properly populated.

In some embodiments, some of the fields may be pre-populated for the PR based on information contained in the initial contact email or text communication generated by the PhrmaConnect software (e.g., any one or more of fields 701, 703, 707, 709, and 711).

In addition, the information entered in each of the aforementioned fields is stored as part of the record file for this interaction and is maintained in a database that is searchable by any of these fields. Thus, for instance, a pharmaceutical company can easily find any record pertaining to any particular medication, PR, HCP, condition, campaign, etc. (or any combination of two or more of the fields).

FIG. 8 is an exemplary embodiment of an email that may be sent to each of the invitees for a meeting created and scheduled in accordance with the description above.

Once scheduled, the meeting will occur through the DxConnect software. Particularly, DxConnect includes a full-featured video conferencing suite with enhanced security and including the capability for the PR to show many forms of media, including video files, audio files, audio/video files, text documents (e.g., studies, medical research, articles, etc.), photographs, brochures, etc. In some embodiments, the system allows the PR to permit the HCP, at the PR's discretion, to download a copy of any of them. In some embodiments, the PR may be enabled to permit the HCPs to download other documents or files that may not have been displayed during the meeting. In an embodiment, the download must occur during the meeting so that it can be properly recorded that the download occurred.

In some preferred embodiments, the system makes a complete video and audio recording of the conference.

In some embodiments, the media that can be shared with and/or downloaded by the HCP is limited to a set of media that is preconfigured in the HPIS. In this manner, the pharmaceutical company can be assured that only pre-approved documentation (or other media) is presented, shared, or made available to the HCPs.

In addition, the HPIS also creates and stores in a searchable database a file record of all clicks during the meeting, all documents, videos, photographs, or other media shared/displayed during the meeting, and all documents, videos, photographs, or other media downloaded by the HCP. In some embodiments, the record even includes data as to how long any given file was shared (e.g., by including a start and stop time of any such sharing).

The recordkeeping of all clicks, downloads, and sharing of media is an extremely useful feature insofar as it allows easier searching of the file records for specific documents or other media that was shared, downloaded, or clicked on than would be possible if only an audio/video recording of the meeting existed.

In some embodiments, the PR may add any relevant notes to the record after the meeting is concluded. Merely a few examples of the types of information that a PR might want to record via such notes include whether the HCP requested samples, questions from the HCP that the PR was unable to answer (and that may need a follow up conference), a level of perceived interest of the HCP(s) during the meeting, or any other piece of information that a pharmaceutical company or PR may be interested in having in a searchable database of such meetings.

These file records of all meetings conducted via the DxConnect software suite are maintained in a searchable database. In this manner, a pharmaceutical company can search through the database to find all conferences meeting any number of criteria, such as conferences in which a certain document, video, or other file was shared or conferences in which a certain document, video, or other file was downloaded by the HCP (and which HCP, if the meeting includes multiple HCPs),

Once the meeting is ended, the HPIS generates a detailed report of the meeting and makes it available to the PR. In some embodiments, the record may also be made available to the HCP either automatically or at the request of the PR.

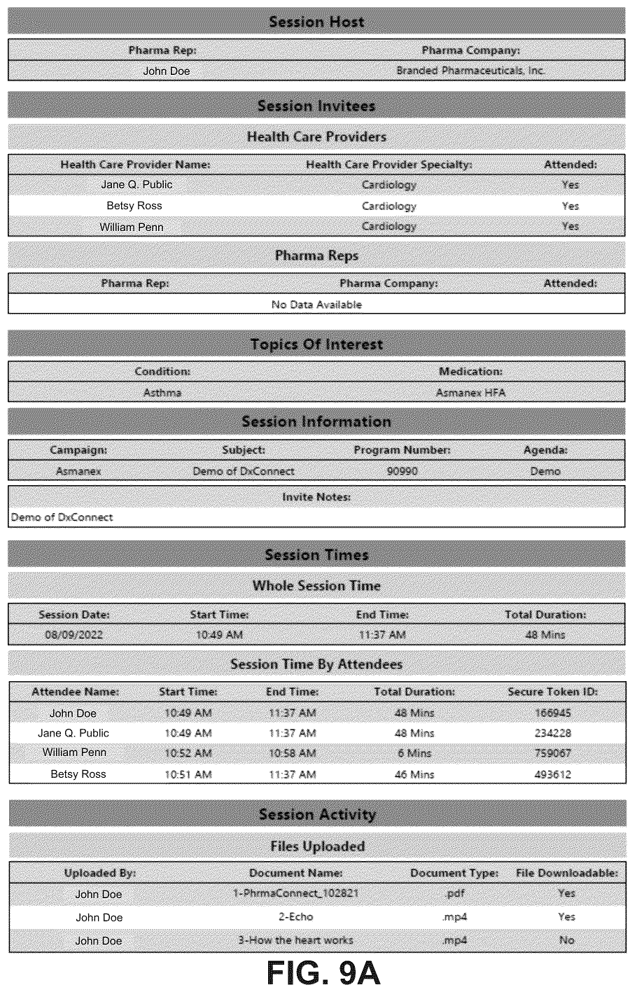

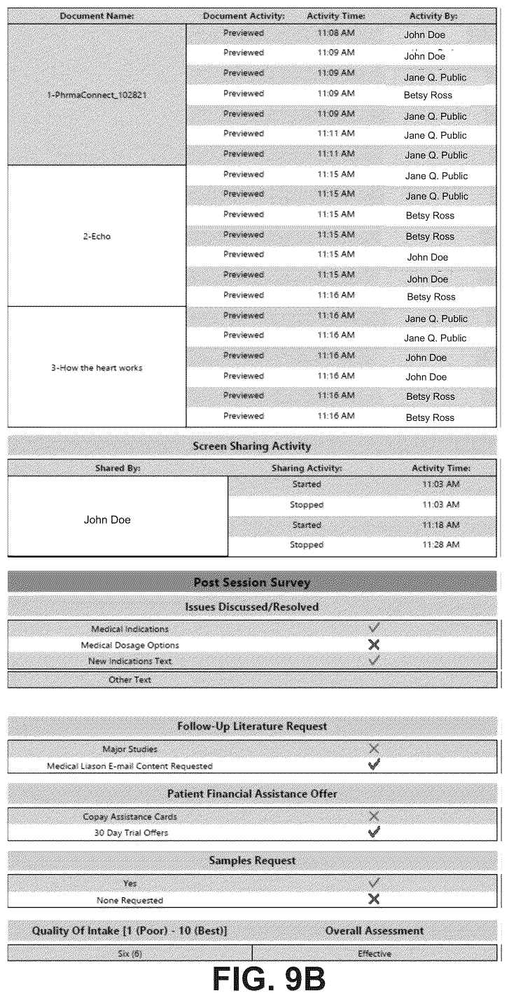

FIGS. 9A and 9B show an example of such a report. As can be seen, the report is largely self-explanatory and includes the identities of the attendees, broken down into the identities of (1) the host PR, (2) the HCP attendees, and (3) any other PRs invited to attend. Next is a Topics of Interest section that identifies the condition and/or medication of interest followed by a Session Information section that identifies any relevant Campaign, Subject, Program Number, and/or Agenda.

The information for populating all of the above-noted fields may be obtained from the data entered during the creation of the invitation (see FIG. 7).

The next section, Session Times, discloses the total session time, including the Date, Start Time, End Time, and Duration. It further includes a separate sub-section, Session Time By Attendees, which includes, for each attendee, the name of the attendee followed by his/her Start Time, End Time, Duration, and Secure Token ID. The Secure Token ID may be a unique passcode sent to each attendee that the attendee must enter into a welcome screen before being admitted to the conference session (also referred to in the item labelled 2 in the email of FIG. 8, for instance).

The next section, Session Activity, includes a sub-section, Files Uploaded, comprises a list of all documents uploaded by the host during the meeting. This includes the identity of the person who uploaded the document, the name of the document, the document type and whether the document was made available for download by the attendees. In an embodiment, the host has full control of the documents to be displayed or uploaded unless the host chooses to give control to a participant during the meeting. Control can be retaken by the host at any time.

In addition, as seen in FIG., 9B, this subsection also includes a more detailed report for each uploaded document showing (1) Document activity, the time at which the activity occurred, and who performed the activity. Some exemplary types of document activities include showing the specific document on the display (referred to as “preview”) and downloading a copy of the specific document that has been displayed.

Another sub-section of the Session Activity section is Screen Sharing Activity. This section discloses who shared their screens during the meeting, including start time and stop time for each sharing segment.

There also is a Post Session Survey segment that the Rep can manually fill out after (or during) the meeting. It comprises several subsections, including an Issues Discussed/Resolved subsection, in which the Rep host may list any issues discussed during the meeting and add a checkmark or X to indicate whether the issue was resolved during the meeting.

The report also may include a Follow-Up Literature Request sub-section, in which the Rep host may list any literature requested by an attendee (or deemed by the Rep to be potentially of interest to an attendee) and further including a field for adding a checkmark or X which may be used to indicate whether the Rep provided the requested literature to the attendee(s). It further may include a Patient Financial-Assistance Offer sub-section, in which the Rep may list any such incentives and whether or not they were offered. It further may include a Samples Request sub-section, in which the Rep host may indicate whether the attendee(s) requested samples.

Finally, in the last sub-section, the Rep may input his or her overall assessment of the conference. In some embodiments, this may include a field for inputting a numerical rating of the Rep's perceived quality of the attendee's intake of the information provided (e.g., on a scale of 1 to 10) as well as an overall assessment of the meeting.

The HPIS 110 comprises two distinct software packages. Particularly, PhrmaConnect 110a is the portion that handles the HCPs initial interface with the system and handles at least a part of the initial request from the HCP to be contacted by a Rep, including any request for samples of a medication (120) as discussed above in connection with FIG. 3.

Referring back to FIG. 1 for a synopsis of the HPIS as described hereinabove, an HCP 107 submits an initial request to be contacted by a Rep through a PhrmaConnect GUI, which may be integrated with the HCP's EHR or EPDS software suite.

PhrmaConnect is adapted to run on laptops as well as mobile devices (111) (including cellular telephones and tablet devices) so that HCPs can access the HPIS 110 features from almost anywhere using almost any electronic communication device.

Second, DxConnect 110b is the interface between the Rep 108 and the system 110 and handles (1) all further scheduling of conferences between the HCP and the Rep (112), (2) provides the interactive video conferencing for the meeting (114), (3) allows for sharing of documents, photographs, videos, and other materials during the meeting (displaying the materials via the video conferencing) (116), (4) downloading of any such materials by the HCP at the PR's discretion (118), (5) allows the HCP to request samples of any medications (either via a button in one of the user interfaces or by requesting it of the PR (and the PR may note the request in his/her post-meeting notes)) 120, (6) generates a detailed report of the meeting after the completion of the meeting (124), and (7) Customer Relationship Manager (CRM) software integration (126) . . . . CRM software is commonly used in this field and is a software product that allows PRs to record and keep track of their detailed interactions with healthcare providers, whether in person, by phone, or via web meetings. CRM software also commonly provides the PR a documented appointment scheduler. The DxConnect and PhrmaConnect software integrates with numerous CRMs where the web interactions between PRs and HCPs are documented and memorialized in the CRM.

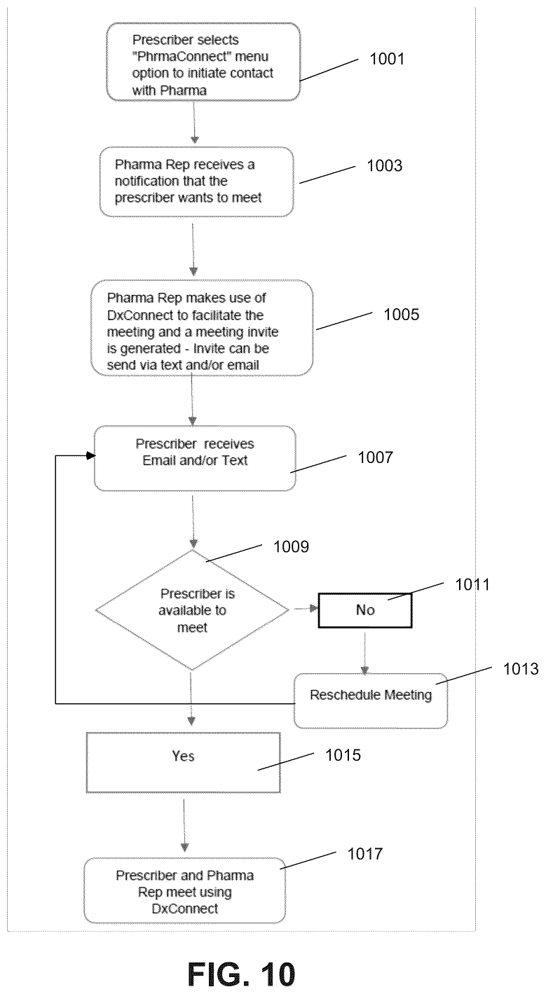

FIG. 10 is a flowchart illustrating a sequence of operations in accordance with one particular embodiment of the overall process described hereinabove. Particularly, at 1001, when an HCP wishes to obtain more information about a medication, the HCP selects the PhrmaConnect button on the appropriate user interface (see, e.g., FIGS. 2, 3A, and 3B, for example). This activates the PhrmaConnect software, which allows the HCP to enter the necessary information (e.g., what medication the HCP is interested in) and cause the PhrmaConnect software to generate an email or text message to an appropriate Rep, e.g., as a function of the HCP's geographic location and the particular medication of interest.

At 1003, the PhrmaConnect software sends the email or text message (such as shown in FIG. 5) to the selected Rep.

At 1005, the Rep activates the DxConnect software to schedule a meeting with the HCP such as described and shown above in connection with FIGS. 6-8.

At 1007, the HCP receives an email (e.g., FIG. 8) or text message proposing a date and time for the meeting. At 1009, the HCP determines whether or not to accept the meeting invitation. If the HCP cannot make the meeting (or has changes his/her mind), the HCP may reply to the email or text meeting invitation requesting a different time (1011). In response, the Rep may send out a revised meeting invitation (1013), and the flow will return to 1007).

When the HCP determines that the meeting date and time is acceptable, the HCP may reply to the email or text accepting the meeting invitation (1015).

At 1017, the Rep and HCP meet using the video conferencing portion of the DxConnect software.

It should be understood that the embodiments presented above focusing on prescription medications are merely exemplary. The systems disclosed herein may be implemented within other contexts, such as to permit an HCP to connect with a Rep relating to medical instruments, treatments, and/or non-prescription drugs. Other applications of the system include uses for conducting and recording interactions between patients/subjects of medical clinical trials and the relevant professionals (doctors, pharmaceutical researchers, etc.). The system may even be used to facilitate and record interactions between physicians (or other health care professionals) and patients.

Yet another application is to facilitate and record interactions between patients and nurses/HCPs that are located in call centers. For example, it is common for pharmaceutical and medical device companies to run call centers for fielding calls from potential and/or existing users of their medications or other medical devices that may have questions about a medication they are taking, a medical device that they are using, and/or a procedure that they are following. For example, many patients administer drugs to themselves via intravenous injection and sometimes need training on self-administering an injectable medication, which training may be obtained via calling in to such a call center.

In yet other embodiments, the system may be used to facilitate and record medical roundtable discussions between physicians located remotely from each other.

It should be noted that the PhrmaConnect and DxConnect portions of the overall HPIS package may be used entirely independently of each other. That is, a Rep may set up and conduct any conference with an HCP using DxConnect. There is no requirement that the meeting request has to have been received via PhrmaConnect. Likewise, a Rep may choose to conduct a conference in person with an HCP that requested a meeting via PhrmaConnect (or even remotely using a different video conferencing system or a conventional telephone call). Of course, that is not recommended insofar as doing so would deprive the Rep and the pharmaceutical company of all of the afore-described features, benefits, and record-keeping of the DxConnect system.

Having thus described a few particular embodiments of the invention, various alterations, modifications, and improvements will readily occur to those skilled in the art. Such alterations, modifications and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer readable medium for execution by a computer or processor. Examples of non-transitory computer-readable storage media include, but are not limited to, a read only memory (ROM), random access memory (RAM), a register, cache memory, semiconductor memory devices, magnetic media such as internal hard disks and removable disks, magneto-optical media, and optical media such as CD-ROM disks, and digital versatile disks (DVDs).

Moreover, in the embodiments described above, processing platforms, computing systems, controllers, and other devices containing processors are noted. These devices may contain at least one Central Processing Unit (“CPU”) and memory. In accordance with the practices of persons skilled in the art of computer programming, reference to acts and symbolic representations of operations or instructions may be performed by the various CPUs and memories. Such acts and operations or instructions may be referred to as being “executed,” “computer executed” or “CPU executed.”

One of ordinary skill in the art will appreciate that the acts and symbolically represented operations or instructions include the manipulation of electrical signals by the CPU. An electrical system represents data bits that can cause a resulting transformation or reduction of the electrical signals and the maintenance of data bits at memory locations in a memory system to thereby reconfigure or otherwise alter the CPU's operation, as well as other processing of signals. The memory locations where data bits are maintained are physical locations that have particular electrical, magnetic, optical, or organic properties corresponding to or representative of the data bits. It should be understood that the exemplary embodiments are not limited to the above-mentioned platforms or CPUs and that other platforms and CPUs may support the provided methods.

The data bits may also be maintained on a computer readable medium including magnetic disks, optical disks, and any other volatile (e.g., Random Access Memory (“RAM”)) or non-volatile (e.g., Read-Only Memory (“ROM”)) mass storage system readable by the CPU. The computer readable medium may include cooperating or interconnected computer readable medium, which exist exclusively on the processing system or are distributed among multiple interconnected processing systems that may be local or remote to the processing system. It is understood that the representative embodiments are not limited to the above-mentioned memories and that other platforms and memories may support the described methods.

In an illustrative embodiment, any of the operations, processes, etc. described herein may be implemented as computer-readable instructions stored on a computer-readable medium. The computer-readable instructions may be executed by a processor of a mobile unit, a network element, and/or any other computing device.

There is little distinction left between hardware and software implementations of aspects of systems. The use of hardware or software is generally (but not always, in that in certain contexts the choice between hardware and software may become significant) a design choice representing cost vs. efficiency tradeoffs. There may be various vehicles by which processes and/or systems and/or other technologies described herein may be effected (e.g., hardware, software, and/or firmware), and the preferred vehicle may vary with the context in which the processes and/or systems and/or other technologies are deployed. For example, if an implementer determines that speed and accuracy are paramount, the implementer may opt for a mainly hardware and/or firmware vehicle. If flexibility is paramount, the implementer may opt for a mainly software implementation. Alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples may be implemented, individually and/or collectively, by a wide range of hardware, software, firmware, or virtually any combination thereof. Suitable processors include, by way of example, a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Application Specific Standard Products (ASSPs); Field Programmable Gate Arrays (FPGAs) circuits, any other type of integrated circuit (IC), and/or a state machine.

The present disclosure is not to be limited in terms of the particular embodiments described in this application, which are intended as illustrations of various aspects. Many modifications and variations may be made without departing from its spirit and scope, as will be apparent to those skilled in the art. No element, act, or instruction used in the description of the present application should be construed as critical or essential to the invention unless explicitly provided as such. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods or systems.

It is also to be understood that the terminology used herein is for the purpose of describing particular embodiments only, and is not intended to be limiting.

In certain representative embodiments, several portions of the subject matter described herein may be implemented via Application Specific Integrated Circuits (ASICs), Field Programmable Gate Arrays (FPGAs), digital signal processors (DSPs), and/or other integrated formats. However, those skilled in the art will recognize that some aspects of the embodiments disclosed herein, in whole or in part, may be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein may be distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc., and a transmission type medium such as a digital and/or an analog communication medium (e.g., a fiber optic cable, a waveguide, a wired communications link, a wireless communication link, etc.).

The herein described subject matter sometimes illustrates different components contained within, or connected with, different other components. It is to be understood that such depicted architectures are merely examples, and that in fact many other architectures may be implemented which achieve the same functionality. In a conceptual sense, any arrangement of components to achieve the same functionality is effectively “associated” such that the desired functionality may be achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, where only one item is intended, the term “single” or similar language may be used. As an aid to understanding, the following appended claims and/or the descriptions herein may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”). The same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, the terms “any of” followed by a listing of a plurality of items and/or a plurality of categories of items, as used herein, are intended to include “any of,” “any combination of,” “any multiple of,” and/or “any combination of multiples of” the items and/or the categories of items, individually or in conjunction with other items and/or other categories of items. Moreover, as used herein, the term “set” or “group” is intended to include any number of items, including zero. Additionally, as used herein, the term “number” is intended to include any number, including zero.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein may be readily broken down into a lower third, middle third and upper third, etc. As will also be understood by one skilled in the art all language such as “up to,” “at least,” “greater than,” “less than,” and the like includes the number recited and refers to ranges which can be subsequently broken down into subranges as discussed above. Finally, as will be understood by one skilled in the art, a range includes each individual member. Thus, for example, a group having 1-3 items refers to groups having 1, 2, or 3 items. Similarly, a group having 1-5 items refers to groups having 1, 2, 3, 4, or 5 items, and so forth.

Moreover, the claims should not be read as limited to the provided order or elements unless stated to that effect. In addition, use of the terms “means for” in any claim is intended to invoke 35 U.S.C. § 112, ¶6 or means-plus-function claim format, and any claim without the terms “means for” is not so intended.

Although the invention is illustrated and described herein with reference to specific embodiments, the invention is not intended to be limited to the details shown. Rather, various modifications may be made in the details within the scope and range of equivalents of the claims and without departing from the invention.

Throughout the disclosure, one of skill understands that certain representative embodiments may be used in the alternative or in combination with other representative embodiments.

1. A computer-readable device for a pharmaceutical representative (PR) to interface with a health care provider (HCP) via electronic communications about a medication, the device comprising non-transitory instructions, which, when executed by a processor, cause the processor to perform operations, the operations comprising:
receiving an electronic communication identifying an HCP, a medication, and an electronic address for communicating with the HCP;
responsive to receiving the electronic communication, generating a first graphical user interface (GUI) configured for scheduling an electronic meeting between the HCP and a PR, the first GUI comprising at least (1) a field for populating with an identity of the PR, (2) a field for populating with an identity of the HCP, (3) a field for populating with the medication, (4) a field for populating with a time for a meeting between the PR and the HCP;
responsive to at least each of the four fields being populated, transmitting to the HCP an invitation to an electronic meeting between the HCP and the PR;
creating an electronic meeting environment in which the PR and HCP may conduct the scheduled electronic meeting, the environment including at least capabilities for audio and video communication between the HCP and the PR via a communication network, capability for uploading files for the meeting, and file sharing capabilities;
recording the meeting, including recording at least audio of the meeting, video of the meeting, files uploaded in the meeting, files downloaded by the HCP in the meeting, and an identity of each participant in the meeting;
storing in a database at least the time of the meeting, a duration of the meeting, an identity of the medication, the participants in the meeting, the files uploaded in the meeting, and the files downloaded by the HCP in the meeting; and
searching the database by any one or more of the fields.

2. The device of claim 1 wherein the electronic address comprising at least one of an email address and a telephone number and wherein the electronic communication comprises at least one of an email and a text message.

3. The device of claim 1 wherein the operation of recording the meeting further comprises recording at least one of the start time of the meeting, the end time of the meeting, an advertising campaign corresponding to the medication, a medical condition relevant to the medication, a time at which each file was uploaded during the meeting and by which meeting participant, a time at which each file was shared during the meeting and by which meeting participant, a time at which each instance of screen sharing occurred during the meeting and by which participant, a time at which each file was downloaded by the HCP and an identity of the downloading HCP.

4. The device of claim 3 wherein the operation of storing further comprises storing in the database at least one of the start time of the meeting, the end time of the meeting, an advertising campaign corresponding to the medication, a medical condition relevant to the medication, a time at which each file was uploaded during the meeting and by which meeting participant, a time at which each file was shared during the meeting and by which meeting participant, a time at which each instance of screen sharing occurred during the meeting and by which participant, a time at which each file was downloaded by the HCP and an identity of the downloading HCP.

5. The device of claim 1 wherein the operations further comprise:
generating a user interface after the meeting is ended in which the PR can enter additional fields of information including at least one of (1) an identity of any literature requested during the meeting, (2) whether the HCP requested a sample of the medication, and (3) whether the PR offered any patent financial assistance offers to the HCP.

6. The device of claim 1 wherein the operations further comprise:
providing capabilities to search the database for meetings by any of the fields.

7. The device of claim 1 wherein the electronic communication further comprises an indication of whether the HCP is requesting a sample of the medication.

8. A computer-readable device for a pharmaceutical representative (PR) to interface with a health care provider (HCP) via electronic communications about a medication, the device comprising non-transitory instructions, which, when executed by a processor, cause the processor to perform operations, the operations comprising:
generating a graphical user interface in which a health care provider (HCP) may select a medication and request to be contacted by a pharmaceutical representative (PR) about the medication;
responsive to the request, searching a first database of PRs and the medications the PRs are permitted to represent to identify a PR suitable to the request as a function of at least the selected medication;
generate an electronic communication identifying the requesting HCP and the selected medication; and
transmitting the electronic communication to the identified PR.

9. The device of claim 8 wherein the device is integrated in an Electronic Prescription Dispensing System (EPDS).

10. The device of claim 9 wherein the operations further comprise:
searching a second database of HCPs and at least their geographic locations to determine a geographic location of the requesting HCP; and
wherein the identifying the suitable PR is further based on a geographic location of the HCP.

11. The device of claim 9 wherein the operations further comprise:
searching a second database of HCPs and at least their geographic locations to determine a geographic location of the requesting HCP; and
wherein the first database includes a geographic location that each PR covers and whether identifying the suitable PR is further based on the geographic location that each PR covers.

12. The device of claim 8 wherein the operation of generating the graphical user interface in which the HCP may select a medication and request to be contacted by a PR about the medication comprises displaying a list of featured medications.

13. The device of claim 12 wherein the featured medications are based on at least one of statistics as to a prevalence of use of a medication, statistics as to a prevalence of patient inquiries about a medication, a geographic location of the HCP, a prevalence of a medical condition, novelty of a medication, sponsorship of a medication by a pharmaceutical company, and current events.

14. The method of claim 8 wherein the operations further comprise:
generating a graphical user interface indicating to the requesting HCP that a request for a meeting has been transmitted to a PR.

15. A method for a pharmaceutical representative (PR) to interface with a health care provider (HCP) about a medication via electronic communications, the method comprising:
receiving an electronic communication identifying an HCP, a medication, and an electronic address for communicating with the HCP;
responsive to receiving the electronic communication, generating a first graphical user interface (GUI) configured for scheduling an electronic meeting between the HCP and a PR, the first GUI comprising at least (1) a field for populating with an identity of the PR, (2) a field for populating with an identity of the HCP, (3) a field for populating with the medication, (4) a field for populating with a time for a meeting between the PR and the HCP;
responsive to at least each of the four fields being populated, transmitting to the HCP an invitation to an electronic meeting between the HCP and the PR;
creating an electronic meeting environment in which the PR and HCP may conduct the scheduled electronic meeting, the environment including at least audio and video communication between the HCP and the PR via a communication network, capability for uploading files for the meeting, and file sharing capabilities;
recording the meeting, including recording at least audio of the meeting, video of the meeting, files uploaded in the meeting, files downloaded by the HCP in the meeting, and an identity of each participant in the meeting;
storing in a database at least the time of the meeting, a duration of the meeting, an identity of the medication, the participants in the meeting, the files uploaded in the meeting, and the files downloaded by the HCP in the meeting; and
searching the database by any one or more of the fields.

16. The method of claim 15 wherein the electronic address comprising at least one of an email address and a telephone number and wherein the electronic communication comprises at least one of an email and a text message.

17. The method of claim 15 wherein recording the meeting further comprises recording at least one of the start time of the meeting, the end time of the meeting, an advertising campaign corresponding to the selected medication, a medical condition relevant to the selected medication, a time at which each file was uploaded during the meeting and by which meeting participant, a time at which each file was shared during the meeting and by which meeting participant, a time at which each instance of screen sharing occurred during the meeting and by which participant, a time at which each file was downloaded by the HCP and an identity of the downloading HCP.

18. The method of claim 17 wherein the storing further comprises storing in the database at least one of the start time of the meeting, the end time of the meeting, an advertising campaign corresponding to the selected medication, a medical condition relevant to the selected medication, a time at which each file was uploaded during the meeting and by which meeting participant, a time at which each file was shared during the meeting and by which meeting participant, a time at which each instance of screen sharing occurred during the meeting and by which participant, a time at which each file was downloaded by the HCP and an identity of the downloading HCP.

19. The method of claim 8 further comprising:
generating a user interface after the meeting is ended in which the PR may enter additional fields of information including at least one of (1) an identity of any literature requested during the meeting, (2) whether the HCP requested a sample of the medication, and (3) whether the PR offered any patent financial assistance offers to the HCP.

20. The method of claim 8 wherein the operations further comprise:
providing capabilities to search the database for meeting by any of the fields.
